(19) United States
(12) Patent Application Publication
(10) Pub. No.: US 2010/0130983 A1
(43) Pub. Date: May 27, 2010

(54) Drill Guide for Angled Trajectories

(75) Inventors: Lisa R. Thornhill, Dallas, TX (US); Viorel Mocanu, Lewisville, TX (US)

Correspondence Address: BAKER BOTTS L.L.P., 2001 ROSS AVENUE, SUITE 600, DALLAS, TX, 75201-2980, US

(73) Assignee: OsteoMed L.P., Addison, TX (US)

(21) Appl. No.: 12/324,142

(22) Filed: Nov. 26, 2008

(51) Int. Cl. A61B 17/58

(52) U.S. Cl. 606/96

(57) ABSTRACT

A system for establishing a trajectory for a pilot hole includes a plate comprising a screw hole defined by a rim and an inner surface surrounding the screw hole, and a drill guide comprising a body having a guide well formed therein, the guide well defined by a wide opening in the body disposed opposite a narrow opening in the body and an interior surface that tapers from the wide opening to the narrow opening, the narrow opening surrounded by a tip of the body, the tip configured to fit into the screw hole.

### TECHNICAL FIELD

The present disclosure relates generally to repairing bone fractures, and more particularly, to a drill guide for angled trajectories.

### BACKGROUND

When repairing a broken, fractured, or shattered bone, a physician may often be faced with the task of affixing a fixation plate to the bone in order to align the bone, and possibly, to hold bone fragments together. In order to affix the fixation plate to the bone, the surgeon may screw a bone screw into a predrilled pilot hole in the bone through one of a plurality of screw holes in the fixation plate. Since numerous screw holes may be spread out across the entirety of the fixation plate, the surgeon may affix virtually any portion of the fixation plate to the bone by inserting a suitable number of bone screws through the fixation plate and into the bone.

To prevent the bone screws from backing out of the fixation plate once inserted, the inner surface of each screw hole may include a set of locking threads configured to interfere with a corresponding set of locking threads on the head of each bone screw. Consequently, when a bone screw is screwed into one of the threaded screw holes in the fixation plate, the locking threads in the screw hole and/or the locking threads on the head of the bone screw may deform to lock the bone screw into the fixation plate.

In certain cases, proper placement and positioning of the fixation plate may call for inserting a bone screw into the fixation plate at an angle other than parallel to the central axis of the screw hole. For example, if the underlying bone beneath a particular screw hole is weak, for example, due to its proximity to a fracture line, the surgeon may wish to angle the bone screw away from the fracture line so as to anchor the bone screw into a more solid bony mass. In another scenario, the surgeon may wish to avoid a nerve underlying the screw hole.

### SUMMARY

In particular embodiments, the present disclosure provides for a system and method for establishing a trajectory for a pilot hole. The system may include a plate having a screw hole. The screw hole may be defined by a rim and an inner surface surrounding the screw hole. The system may further include a drill guide comprising a body having a guide well formed therein. The guide well may be defined by a wide opening in the body disposed opposite a narrow opening in the body and an interior surface that tapers from the wide opening to the narrow opening. The narrow opening may be surrounded by a tip of the body that is configured to fit into the screw hole.

A method for establishing a trajectory for a pilot hole may include placing a plate onto a bone, the plate including a screw hole defined by a rim and an inner surface surrounding the screw hole. The method may further include fitting a drill guide into the screw hole, the drill guide comprising a body having a guide well formed therein. The guide well may be defined by a wide opening in the body disposed opposite a narrow opening in the body and an interior surface that tapers from the wide opening to the narrow opening. The narrow opening may be surrounded by a tip of the body that is configured to fit into the screw hole.

In particular embodiments, the method may further include inserting a drill bit into the guide well and drilling a pilot hole into the bone while keeping the drill bit within the confines of the interior surface.

Technical advantages of particular embodiments of the present disclosure may include providing a system and method for enabling a surgeon to effectively judge the maximum angle of insertion for which the locking effect between a locking bone screw and a locking fixation plate will be maintained. This technical advantage may be realized through the use of a drill guide placed on the fixation plate that physically defines the maximum angle of insertion, giving the surgeon a defined range of acceptable angles within which to drill a pilot hole. The drill guide may further include a tip that fits securely into the screw hole of the fixation plate to align the drill guide with the screw hole and to protect the threading inside the screw hole from the drill bit during creation of a pilot hole, yet another technical advantage.

Other technical advantages of the present disclosure will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 illustrates a system for establishing a trajectory for a pilot hole according to an example embodiment of the present disclosure;

FIG. 2 illustrates an isometric view of an example embodiment of a fixation plate according to an example embodiment of the present disclosure;

FIG. 3 illustrates an isometric view of an example embodiment of a drill guide according to an example embodiment of the present disclosure; and

FIG. 4 illustrates an example cross section view of a portion of the drill guide of FIG. 3 exposing a more detailed view of the interior surface of the drill guide.

### DETAILED DESCRIPTION OF EXAMPLE EMBODIMENTS

Various fixation plates may include specially designed screw holes that may lockably engage a bone screw inserted (e.g., “screwed in”) along a trajectory other than parallel to (e.g., coaxial with) the central axis of such screw holes. In one example embodiment, the bone screw may include threading on the underside of the head that interferes with threading inside the screw hole to lock the bone screw into the fixation plate once the bone screw is screwed into the screw hole.

To facilitate insertion of the bone screw into the screw hole along an angled trajectory, a pilot hole may be drilled into the bone to establish the trajectory for the bone screw. After the pilot hole has been created, the tip of the bone screw may be inserted into the pilot hole through the screw hole in the fixation plate and rotated until the threaded portion on the underside of the head comes to bear on the threaded portion of the screw hole. At this point, further rotation of the bone screw may cause the threaded portion of the head to interfere with the threading inside the screw hole and lock the bone screw into the plate.

Depending upon design, the above-described locking effect between the bone screw and the fixation plate may only be effective up to a maximum angle of insertion of the bone screw relative to the central axis of the screw hole. If the trajectory of the bone screw exceeds the maximum angle of insertion, the locking effect may become unreliable or may fail, increasing the propensity of the bone screw to back out of the screw hole. In other words, depending upon design, the screw hole in the fixation plate may only be able to accommodate a certain angular range of screw engagement before the locking mechanism is weakened or rendered inoperable.

In the operating room, a surgeon may not be able to effectively judge the maximum angle of insertion by visual inspection and may need a tool to help him drill a pilot hole within the specified limits of the plate design (e.g., within the maximum angle of insertion). Consequently, there is a need for a system and method to reliably establish the trajectory for the pilot hole within the maximum angle of insertion and also a need to protect the threading inside the threaded screw hole from being deformed by the drill bit during creation of the pilot hole.

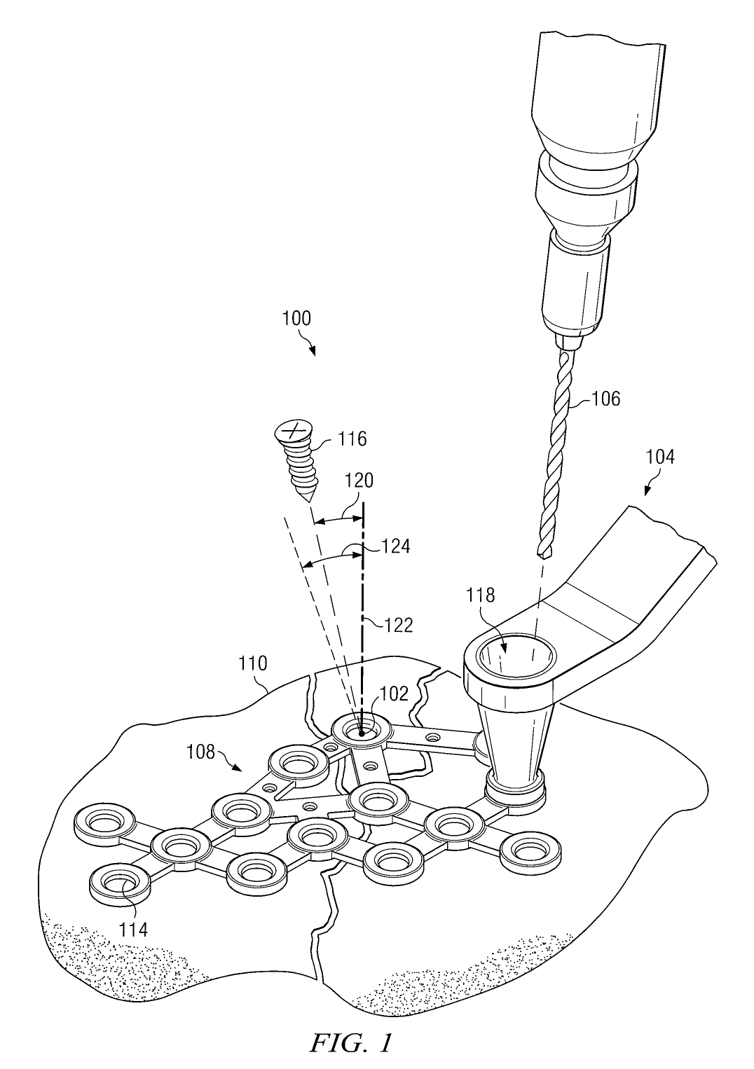

FIG. 1 illustrates a system 100 for establishing a trajectory for a pilot hole 102 according to an example embodiment of the present disclosure. System 100 generally includes a drill guide 104 for guiding a drill bit 106 and a fixation plate 108 for affixing portions of a bone 110 together and for holding drill guide 104 steady relative to bone 110.

In the pictured embodiment, system 100 is being used relative to a single fractured bone 110; however, particular embodiments of system 100 may be applied equally as well to virtually any bone or group of bones in the body. System 100 may also be used to create a pilot hole 102 in a synthetic element such as a surgical implant.

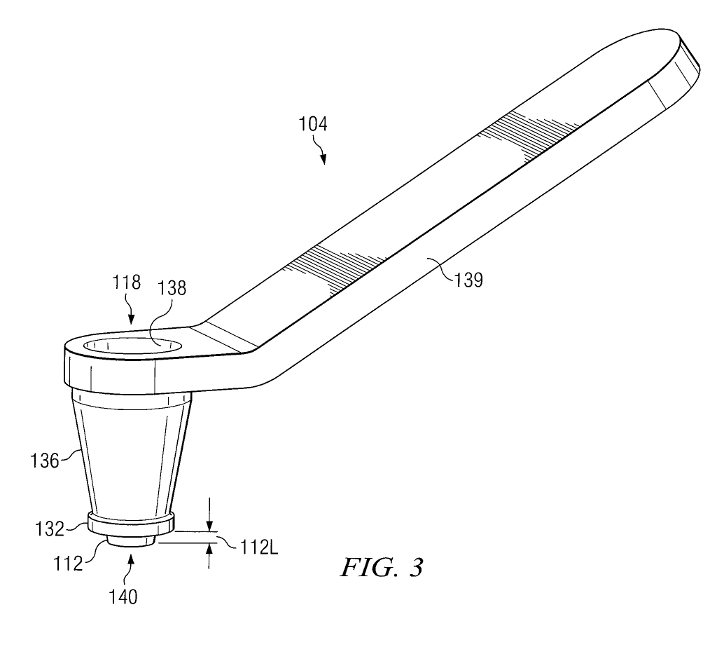

To establish the trajectory for pilot hole 102 using system 100, a surgeon may place fixation plate 108 onto bone 110, after which the surgeon may fit the tip 112 (see FIG. 3) of drill guide 104 into any one of a plurality of screw holes 114 disposed throughout fixation plate 108. In particular embodiments, the surgeon may temporarily or permanently secure fixation plate 108 on bone 110 using pins, bone screws 116, or other suitable means while creating pilot hole 102. In any case, once the tip 112 of drill guide 104 has been inserted into a screw hole 114, the surgeon may create pilot hole 102 by inserting drill bit 106 into a guide well 118 in drill guide 104 and drilling into bone 110 within the confines of guide well 118.

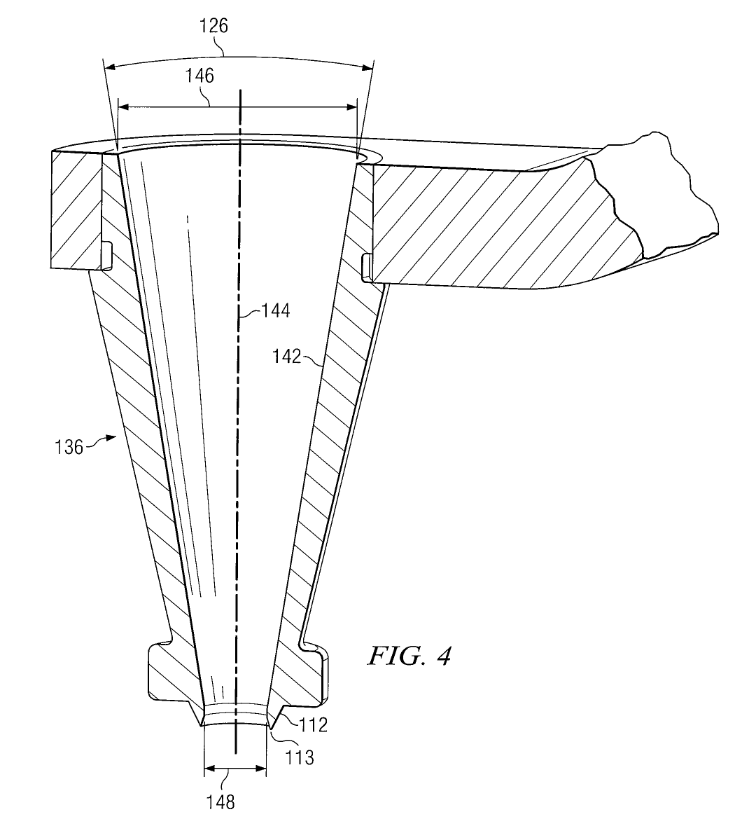

If the surgeon is using a bone screw 116 having a locking feature, drill guide 104 may be used to ensure that the angle of insertion 120 for bone screw 116 (e.g. the trajectory of pilot hole 102 relative to the central axis 122 of screw hole 114) is less than or equal to the maximum angle of insertion 124 for which the desired locking effect between bone screw 116 and fixation plate 108 will be preserved. For example, drill guide 104 may designed such that, once the tip 112 of drill guide 104 has been positioned in a screw hole 114, the aperture angle 126 (see FIG. 4) of guide well 118 is coextensive with the twice the maximum angle of insertion 124. One of ordinary skill in the art will appreciate that aperture angle 126 is coextensive with twice maximum angle of insertion 124 when aperture angle 126 is twice as large as maximum angle of insertion 124 since maximum angle of insertion 124 is measured with respect to central axis 122 while aperture angle 126 is measured with respect to opposing sides of guide well 118. Consequently, by drilling within the confines of drill guide 104, the surgeon may be assured that the angle of insertion 120 established for bone screw 116 is less than or equal to the maximum angle of insertion 124. Once pilot hole 102 has been created using drill guide 104, fixation plate 108 may be affixed to bone 110 by screwing bone screw 116 into pilot hole 102 through screw hole 114.

Depending upon design, drill guide 104, bone screw 116, and fixation plate 108 may be formed from any one or more materials suitable for forming medical devices and implants, such as materials that have high strength-to-weight ratios and that are inert to human body fluids. In certain embodiments, drill guide 104, fixation plate 108, or bone screw 116 may be formed from one or more titanium alloys, which provide several benefits. For example, titanium alloys are relatively lightweight, provide adequate strength for withstanding high forces, are inert to human body fluids, and are visible in radiographs. In particular embodiment, bone screw 116 may be formed from the titanium based alloy Ti6Al4V ELI (per ASTM F136), which provides a desirable combination of benefits, such as those discussed above while fixation plate 108 may be formed from grade 2 or grade 3 titanium (per ASTM F67). In certain other embodiments, bone screw 116 or fixation plate 108 may be formed from one or more resorbable polymers, such as polylactides, polyglycolide, glycolide/lactide copolymers or other copolymers for example, or one or more implantable plastics, such as polyethylene or acetal copolymers for example.

One of ordinary skill in the art will appreciate that the above-described embodiments of system 100 were presented for the sake of explanatory simplicity and will further appreciate that the present disclosure contemplates using any suitable combination and number locking screws 116 and fixation plates 108 to repair bone 110.

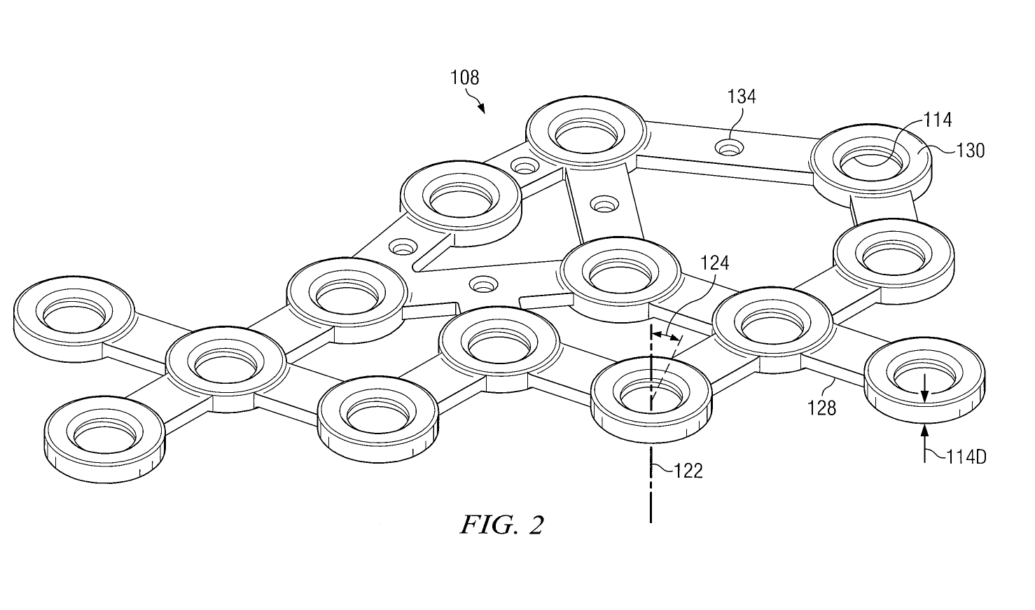

FIG. 2 illustrates an isometric view of an example embodiment of fixation plate 108. For reference purposes, fixation plate 108 (as well as other components of system 100) may be referred to as having a bottom side intended to be placed closest to bone 110 (e.g., to be placed upon bone 110) and a top side intended to be place furthest from bone 110. Though particular features of fixation plate 108 may be explained using such intended placement as a point of reference, this method of explanation is not meant to limit the scope of the present disclosure to any particular configuration of fixation plate 108 or to any particular placement or orientation of fixation plate 108 relative to bone 110 or any other components of system 100.

Fixation plate 108 may typically be any fixture including one or more screw holes 114 for receiving a bone screw 116. In the pictured embodiment, fixation plate 108 generally includes a plurality of screw holes 114 connected to each other in a web-like distribution by a plurality of ribs 128. Each screw hole 114 may include a rim 130 (e.g., a flat surface surrounding screw hole 114). Though in the pictured embodiment, ribs 128 are thinned down relative to each rim 130, particular embodiments of plate 108 may be designed such that the entirety of plate 108 is uniform in thickness. In any case, when drill guide 104 is fitted into a screw hole 114, rim 130 may rest flush against the shoulder 132 of drill guide 104, providing drill guide 104 with a steady foundation on fixation plate 108.

To aid a surgeon in positioning fixation plate 108 relative to bone 110, one or more ribs 128 may comprise a positioning hole 134. As an example and not by way of limitation, a surgeon may insert a K-wire into bone 110 after which the surgeon may position fixation plate 108 on bone 110 by inserting the K-wire through positioning hole 134 and sliding fixation plate 108 down onto bone 110. Additionally, the surgeon may rotate fixation plate 108 about the K-wire using positioning hole 134 to achieve a desired orientation of fixation plate 108 relative to bone 110. Once fixation plate 108 has been properly positioned, the surgeon may use drill bit 106 in conjunction with drill guide 104 to create a pilot hole 102 through screw hole 114. The surgeon may then secure fixation plate 108 to bone 110 by screwing bone screw 116 into screw holes 114 along the trajectory established by pilot hole 102.

Each screw hole 114 may be any an opening in fixation plate 108 configured to accept a bone screw 116. In particular embodiments, the inner surface of each screw hole 114 may be threaded to lockably engage bone screw 116 such that once bone screw 116 has been screwed into screw hole 114, bone screw 116 is prevented from rotating within screw hole 114. As mentioned above, to accomplish this locking feature, the underside of the head of bone screw 116 may include a locking thread configured to interfere with the threading inside screw hole 114. Thus, when bone screw 116 is screwed into screw hole 114, the locking thread on the head of bone screw 116 may deform against the threading inside screw hole 114 to lock bone screw 116 into fixation plate 108.

In particular embodiments, screw holes 114 may be designed to enable bone screw 116 to be screwed in along a trajectory other than parallel to central axis 122 while still maintaining the ability to lockably engage bone screw 116. One example system for achieving an angular locking interface between a bone screw and a fixation plate is described in U.S. Provisional Application 61/106,511, entitled “ANGULATED LOCKING PLATE/SCREW INTERFACE,” filed Oct. 17, 2008.

In particular embodiments, design constraints or other considerations may limit the range of insertion angles for which the locking effect between bone screw 116 and fixation plate 108 remains viable. For example, a manufacturer may design fixation plate 108 such that the locking interface between bone screw 116 and fixation plate 108 remains viable for insertion angles up to ten degrees from parallel the central axis 122 of screw hole 114. Beyond this ten degree radius around central axis 122, the locking effect brought about by the threadable interface between bone screw 116 and plate 108 may be unreliable; for example, the amount of contact between the threading on bone screw 116 and the threading inside screw hole 114 may be insufficient to overcome the mechanical forces that may cause bone screw 116 to back out of screw hole 114. Consequently, the maximum angle of insertion 124 in this example situation is ten degrees. One of ordinary skill in the art will appreciate that the maximum angle of insertion 124 may be defined by the manufacturer according to any suitable criteria. For example, maximum angle of insertion 124 may be defined as the angle beyond which a certain percentage of locking failures occur at a give stress level. In the operating room, drill guide 104 may enable a surgeon to effectively judge the maximum angle of insertion 124 when drilling a pilot hole 102 for bone screw 116 by providing a physical barrier that physically defines maximum angle of insertion 124 for the surgeon. Consequently, by drilling within the confines of drill guide 104, the surgeon may be assured that the trajectory of pilot hole 102 will be less than or equal to the maximum angle of insertion 124.

One of ordinary skill in the art will appreciate that the above-described embodiments of fixation plate 108 were presented for the sake of explanatory simplicity and will further appreciate that the present disclosure contemplates any suitable configuration for fixation plate 108.

FIG. 3 illustrates an isometric view of an example embodiment of drill guide 104. Depending upon design, drill guide 104 may generally include a funnel-shaped body 136 rigidly coupled to a handle 138. Body 136 may have a guide well 118 formed therein that is defined by a wide opening 138 at the top of body 136, a narrow opening 140 at the bottom of body 136, and an interior surface 142 (see FIG. 4) that uniformly tapers (e.g., tapers in diameter) from wide opening 138 to narrow opening 140. In particular embodiments, wide opening 138 and narrow opening 140 may be concentric circles disposed opposite one another on body 136. Body 136 may further include a tip 112 that surrounds narrow opening 140 and a shoulder 132 disposed above tip 112 that includes a flat underside configured to rest flush against rim 130 when tip 112 is inserted into a screw hole 114.

In particular embodiments, drill guide 104 may be fitted into fixation plate 108 by sliding tip 112 into screw hole 114 until shoulder 132 rests flush against the top surface of fixation plate 108 (e.g., the top surface of rim 130). To keep tip 112 from wobbling around in screw hole 114, the outer surface of tip 112 may be generally cylindrical in shape and sized to fit securely (e.g., snugly) into screw hole 116. In particular embodiments, the outer surface of tip 112 may be threaded to enable a surgeon to screw tip 112 into screw hole 116. Tip 112 may be configured to align the central axis 144 of guide well 118 (see FIG. 4) with the central axis 122 of screw hole 114. In particular embodiments, a length 112L of tip 112 may be approximately equal to the depth 114D of screw hole 114 to protect the threading inside screw hole 114 from being deformed by drill bit 106 during the creation of pilot hole 102.

Shoulder 132 may be any fixture or combination of fixtures on the outer surface of body 136 capable of providing a level footing for body 136 relative to fixation plate 108. As an example and not by way of limitation, shoulder 132 may comprise a contiguous smooth flat surface surrounding tip 112. When tip 112 is inserted into screw hole 114, shoulder 132 may abut rim 130 and act as a stop that limits the penetration depth of tip 112 into screw hole 114, and as leveling mechanism that levels body 136 relative to fixation plate 108. As an example and not by way of limitation, the flat underside of shoulder 132 may be disposed perpendicular to the central axis 144 of guide well 118, thereby ensuring the central axis 144 of guide well 118 is parallel to (e.g., coaxial with) the central axis 122 of screw hole 114 when tip 112 is inserted into screw hole 114. Consequently, when drill guide 104 is fitted into a screw hole 114, the outer surface of body 136 may be used to align and couple body 136 with fixation plate 108 while interior surface 142 may be used to limit the insertion angle of drill bit 106 into bone 110.

FIG. 4 illustrates an example cross section view of a portion of drill guide 104 cut through the central axis 144 of guide well 118 to expose a more detailed view of the interior surface 142 surrounding guide well 118. In particular embodiments, interior surface 142 may be defined, in part, by a maximum diameter 146 (e.g., the diameter of wide opening 138), a minimum diameter 148 (e.g., the diameter of narrow opening 140), and an aperture angle 126. Although aperture angle 126 may be chosen according to any criteria, in particular embodiments, guide well 118 may be designed such that aperture angle 126 coincides with twice the maximum angle of insertion 124 for which the locking effect between bone screw 116 and fixation plate 108 is preserved. Thus, if aperture angle 126 coincides with twice the maximum angle of insertion 124, drill bit 106 may be used to create pilot hole 102 along virtually any trajectory within the confines of interior surface 142 while still preserving the desired locking effect between bone screw 116 and fixation plate 108. As an example and not by way of limitation, if the maximum angle of insertion 124 is approximately 10 degrees measured from the central axis 122 of screw hole 112, aperture angle 126 may be approximately 20 degrees. Thus, once drill guide 104 is fitted into screw hole 114, drill bit 106 cannot be inserted through screw hole 114 along any trajectory greater than 10 degrees from central axis 122 without wedging between the portion of interior surface 142 surrounding wide opening 138 and the portion of interior surface 142 surrounding narrow opening 140.

In particular embodiments, to prevent drill bit 106 from grinding against the bottom edge 113 of tip 112 during the creation of pilot hole 102, the portion of interior surface 142 disposed inside tip 112 may have a slight outward taper. In other words, interior surface 142 may come to its narrowest point somewhere in the inside tip 112 (e.g., at the uppermost portion of tip 112) and then grow slightly wider before reaching bottom edge 113. This outward taper may provide clearance for drill bit 106 to be inserted through narrow opening 140 without grinding against bottom edge 113. In this case, narrow opening 140 may refer to the narrowest point of interior surface 142.

One of ordinary skill in the art will appreciate that the above described embodiments of interior surface 142 were presented for the sake of explanatory clarification and will further appreciate that the present disclosure contemplates interior surface 142 having any suitable size or shape. For example, although interior surface 142 was described and illustrated as having a generally circular base yielding a generally right angle conical shape, interior surface 142 could just as easily have a generally square base yielding a generally pyramidal shape or any other geometrically suitable configuration operable to limit the insertion angle of drill bit 106 into bone 110.

Although the present disclosure has been described in several embodiments, a myriad of changes, substitutions, and modifications may be suggested to one skilled in the art, and it is intended that the present disclosure encompass such changes, substitutions, and modifications as fall within the scope of the present appended claims.

What is claimed is:

1. A system for establishing a trajectory for a pilot hole, comprising:
a plate comprising a screw hole defined by a rim and an inner surface surrounding the screw hole; and
a drill guide comprising a body having a guide well formed therein, the guide well defined by a wide opening in the body disposed opposite a narrow opening in the body and an interior surface that tapers from the wide opening to the narrow opening, the narrow opening surrounded by a tip of the body, the tip configured to fit into the screw hole.

2. The system of claim 1, wherein the drill guide further comprises a shoulder disposed adjacent to the tip, the shoulder comprising a flat surface configured to rest on the rim of the screw hole once the tip is fitted into the screw hole.

3. The system of claim 1, wherein a length of the tip is less than or equal to a depth of the screw hole.

4. The system of claim 1, wherein the tip is configured to align a central axis of the guide well with a central axis of the screw hole once the tip is fitted into the screw hole.

5. The system of claim 1, wherein the tip is configured to fit securely into the screw hole.

6. The system of claim 1, wherein the guide well is conical in shape.

7. The system of claim 1, wherein:
the inner surface of the screw hole includes threading configured to lockably engage a bone screw up to a maximum angle of insertion;
the interior surface of the guide well uniformly tapers according to an aperture angle; and
the aperture angle of the guide well is coextensive with twice the maximum angle of insertion of the screw hole once the once the tip is fitted into the screw hole.

8. The system of claim 1, wherein the drill guide further comprises an elongate handle coupled to the body.

9. The system of claim 1, wherein the wide opening is circular, the narrow opening is circular, and the wide opening is concentric with the narrow opening.

10. The system of claim 1, wherein a diameter of a portion of the interior surface disposed inside the tip grows wider as the interior surface extends toward an edge of the tip.

11. A drill guide, comprising a body having a guide well formed therein, the guide well defined by a wide opening in the body disposed opposite a narrow opening in the body and an interior surface that tapers from the wide opening to the narrow opening, the narrow opening surrounded by a tip of the body, the tip configured to fit into a screw hole of a locking plate, the screw hole defined by a rim and an inner surface surrounding the screw hole.

12. The drill guide of claim 11, further comprising a shoulder disposed adjacent to the tip, the shoulder comprising a flat surface configured to rest on the rim of the screw hole once the tip is fitted into the screw hole.

13. The drill guide of claim 11, wherein a length of the tip is less than or equal to a depth of the screw hole.

14. The drill guide of claim 11, wherein the tip is configured to align a central axis of the guide well with a central axis of the screw hole once the tip is fitted into the screw hole.

15. The drill guide of claim 11, wherein the tip is configured to fit securely into the screw hole.

16. The drill guide of claim 11, wherein the guide well is conical in shape.

17. The drill guide of claim 11, wherein:
the inner surface of the screw hole includes threading configured to lockably engage a bone screw up to a maximum angle of insertion;
the interior surface of the guide well uniformly tapers according to an aperture angle; and
the aperture angle of the guide well is coextensive with twice the maximum angle of insertion of the screw hole once the tip is fitted into the screw hole.

18. A method for establishing a trajectory for a pilot hole, comprising:
placing a plate onto a bone, the plate comprising a screw hole defined by a rim and an inner surface surrounding the screw hole; and
fitting a drill guide into the screw hole, the drill guide comprising a body having a guide well formed therein, the guide well defined by a wide opening in the body disposed opposite a narrow opening in the body and an interior surface that tapers from the wide opening to the narrow opening, the narrow opening surrounded by a tip of the body, the tip configured to fit into the screw hole.

19. The method of claim 18, wherein:
the drill guide further comprises a shoulder disposed adjacent to the tip, the shoulder comprising a flat surface configured to rest on the rim of the screw hole once the tip is fitted into the screw hole; and
fitting the drill guide into the screw hole comprises inserting the tip of the body into the screw hole until shoulder abuts the rim of the screw hole.

20. The method of claim 19, further comprising inserting a drill bit into the guide well and drilling a pilot hole into the bone within the confines of the interior surface.

21. The method of claim 20, wherein:
the inner surface of the screw hole includes threading configured to lockably engage a bone screw up to a maximum angle of insertion;
the interior surface of the guide well uniformly tapers according to an aperture angle; and
the aperture angle of the guide well is coextensive with twice the maximum angle of insertion of the screw hole once the tip is fitted into the screw hole.
